(19) United States
(12) Patent Application Publication
(10) Pub. No.: US 2025/0191784 A1
(43) Pub. Date: Jun. 12, 2025

(54) TREATMENT-METHOD SELECTION ASSISTANCE APPARATUS, TREATMENT-METHOD SELECTION ASSISTANCE METHOD, AND COMPUTER-READABLE MEDIUM

(71) Applicants: NEC Corporation, Tokyo (JP); OSAKA UNIVERSITY, Suita-shi, Osaka (JP)

(72) Inventors: Kaori KOBAYASHI, Tokyo (JP); Kenichi KAMIJO, Tokyo (JP); Masanori TOYODA, Tokyo (JP); Masataka KIKUCHI, Osaka (JP)

(73) Assignees: NEC Corporation, Tokyo (JP); OSAKA UNIVERSITY, Suita-shi, Osaka (JP)

(21) Appl. No.: 18/844,708

(22) PCT Filed: Mar. 25, 2022
(86) PCT No.: PCT/JP2022/014199

§ 371 Date: Sep. 6, 2024

(51) Int. Cl. G16H 70/40; G16H 10/60

(52) U.S. Cl. CPC G16H 70/40; G16H 10/60

(57) ABSTRACT

Displaying information on a plurality of patients assists in selecting a treatment method. A treatment-method selection assistance apparatus includes a medication history acquisition unit that acquires a history of administering a medicine to each patient as a medication history, a medical condition acquisition unit that acquires a state of a disease of each patient, and a display control unit that causes a display apparatus to display a display screen on which the medication histories and states of the disease of a plurality of patients are displayed along a time axis.

### TECHNICAL FIELD

The present disclosure relates to a treatment-method selection assistance apparatus, a treatment-method selection assistance method, and a computer-readable medium.

### BACKGROUND ART

Patent Literature 1 discloses an information system that assists analysis of side effects of prescription drugs. Patent Literature 2 discloses a medical information display apparatus that displays various types of medical data on one screen.

### CITATION LIST

### Patent Literature

Patent Literature 1: International Patent Publication No. WO 2015/189958
Patent Literature 2: Japanese Unexamined Patent Application Publication No. 2020-038641

### SUMMARY OF INVENTION

### Technical Problem

When determining a treatment policy for a patient, it is effective to confirm not only a treatment method and efficacy, being selected for the patient so far but also a treatment method and efficacy, being selected for other patients.

Therefore, one of objects that example embodiments disclosed herein intend to achieve is to provide a treatment-method selection assistance apparatus that assists selection of a treatment method by displaying information regarding a plurality of patients.

### Solution to Problem

A treatment-method selection assistance apparatus according to a first aspect of the present disclosure includes: a medication history acquisition means for acquiring a history of administering a medicine to each patient as a medication history; a medical condition acquisition means for acquiring a state of a disease of each patient; and a display control means for causing a display apparatus to display a screen on which the medication histories and states of the disease of a plurality of patients are displayed along a time axis.

A treatment-method selection assistance method according to a second aspect of the present disclosure includes, by a computer: acquiring a history of administering a medicine to each patient as a medication history; acquiring a state of a disease of each patient; and causing a display apparatus to display a display screen on which the medication histories and states of the disease of a plurality of patients are displayed along a time axis.

A non-transitory computer-readable medium according to a third aspect of the present disclosure stores a program for causing a computer to execute: processing of acquiring a history of administering a medicine to each patient as a medication history; processing of acquiring a state of a disease of each patient; and processing of causing a display apparatus to display a display screen on which the medication histories and states of the disease of a plurality of patients are displayed along a time axis.

### Advantageous Effects of Invention

According to the present disclosure, it is possible to assist selection of a treatment method by displaying information regarding a plurality of patients.

### BRIEF DESCRIPTION OF DRAWINGS

FIG. 1 is a block diagram illustrating a configuration of a treatment-method selection assistance apparatus according to a first example embodiment;

FIG. 2 is a schematic diagram illustrating an example of a screen to be displayed;

FIG. 3 is a schematic diagram illustrating an example of a screen to be displayed;

FIG. 4 is a schematic diagram illustrating an example of a screen to be displayed;

FIG. 5 is a schematic diagram illustrating an example of a screen to be displayed; and

FIG. 6 is a schematic diagram illustrating an example of a screen to be displayed.

### EXAMPLE EMBODIMENT

### First Example Embodiment

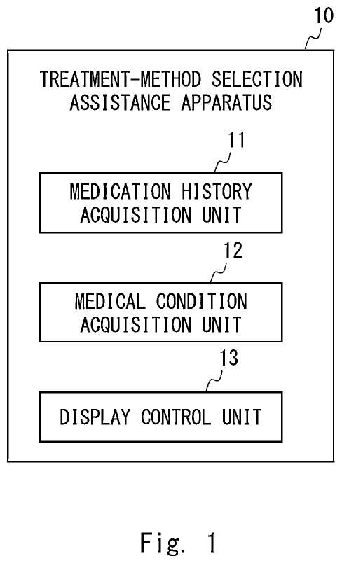

FIG. 1 is a schematic diagram illustrating an example of a configuration of a treatment-method selection assistance apparatus 10 according to a first example embodiment. The treatment-method selection assistance apparatus 10 includes a medication history acquisition unit 11, a medical condition acquisition unit 12, and a display control unit 13. The treatment-method selection assistance apparatus 10 is connected to a display apparatus (e.g., a display or a monitor) which is not illustrated. The treatment-method selection assistance apparatus 10 may include a display apparatus.

The medication history acquisition unit 11 acquires a history of administering a medicine to each patient as a medication history. The medication history includes a name of the medicine administered to each patient and a time period during which the medicine is administered. The medicine may be an anticancer agent.

The medical condition acquisition unit 12 acquires a state of a disease (also referred to as a medical condition) of each patient. When the disease is cancer, the medical condition may be any one of the following: cancer progression (stage), malignancy, class classification, and prognostic factors.

The display control unit 13 causes a display apparatus to display a screen on which the medication histories and the states of the disease of a plurality of patients are displayed along a time axis. The time axis may represent a date and may represent a period from an event to be described below. Since the display screen includes a medication history and a medical condition (e.g., a stage of cancer progression), an effect of treatment by the medicine can be grasped by looking at the screen. Thus, a user (physician) can select a treatment method suitable for the treatment of the patient. The selection of the treatment method is not limited to selecting the medicine to be administered. As a treatment method, it may be selected to perform radiation treatment.

The display control unit 13 may further display an event in which the date is determined for each patient on the screen. In such a case, a mark indicating the event is further displayed along the time axis. The event may be, for example, the start or completion of administration of a medicine. In a case where it is displayed that the administration of the medicine is completed, the display control unit 13 may further display ineffective discontinuation or the end of the plan as a reason for the completion. In addition, the display control unit 13 may display determination of the best overall effect, presence or absence of Grade 3 or more adverse events, and the like. The event may also be that medical care or diagnosis has been performed for each patient, that a specimen has been collected from each patient, that each patient's medical condition (e.g., stage) has changed, and that each patient's illness has been found or registered. The specimen may be tumor tissue or non-tumor tissue. The event may also be a patient's hospitalization and discharge, or a change in a treatment policy or a prescription policy for a medicine.

The event may also be that an expert panel has been held for each patient. The expert panel refers to a meeting in which a plurality of specialists consider an appropriate treatment method for a patient, based on results acquired from cancer gene panel inspection. The treatment-method selection assistance apparatus 10 may be used in an expert panel. By displaying a mark indicating the expert panel on the screen, a user can understand that the treatment policy has changed due to the execution of the expert panel.

The event may also be associated with a degree of response indicating a treatment effect for each patient. An event related to the degree of response is, for example, to perform an evaluation of the degree of response or a test for evaluation of the degree of response. The degree of response of Gastrointestinal Stromal Tumor (GIST) is known as a criterion for evaluating tumor shrinkage. The degree of response represents complete response (CR), partial response (PR), progression (PD: progressive disease), or stability (SD: stable disease). CR represents a state in which the tumor has been completely disappeared. PR represents a state in which the sum of tumor diameters has decreased by 30% or more. SD represents a state in which a size of the tumor does not change. PD represents a state in which the sum of the tumor diameters has increased by 20% or more and an absolute value also has increased by 5 mm or more, or a state in which a new lesion has appeared. The display control unit 13 displays the degree of response on the screen, whereby the user can more accurately grasp the treatment effect of the medicine.

The display control unit 13 may have a function of switching display/non-display of a mark representing each event.

As discussed above, the time axis on the screen to be displayed may represent a time period from the event (e.g., the start of medication treatment). This makes it easy to compare the treatment effects even when the start times of pharmacotherapy and the like are different.

The display control unit 13 may change a first screen in which the time axis represents a date and a second screen in which the time axis represents a time period from the event, depending on a user's selection. By displaying the first screen, it is possible to confirm a date when the event has been performed. Further, by displaying the second screen, even when an occurrence date of the event is different between patients, the treatment effect can be easily compared between the patients. The display control unit 13 may further include a function of changing an event serving as a reference of the time axis depending on a user's selection.

The display control unit 13 may further display a time period during which the medicine is not approved on the screen. The time period during which the medicine is not approved is a time period during which the medicine is unapproved or a time period during which the approval of the medicine has been revoked.

The display control unit 13 may have a function of displaying statistical information relating to a medication period when displaying the medication history. The display control unit 13 may have a function of displaying, together with an average value or a median value of the medication periods, a response rate or the like indicating how many patients the medicine is administered and how many persons is in remission.

The display control unit 13 may further have a function of displaying a date of birth of each patient, a date of registration of each patient's cancer, a medical history of each patient, a medical history of each patient's family, and gene mutation information regarding each patient. A physician can refer to these pieces of information and select a treatment method for each patient. For example, displaying both information regarding a patient to be diagnosed and information regarding a patient having a similar medical history or gene mutation to the patient to be diagnosed on a display apparatus may lead to selection of an appropriate treatment method.

Note that the treatment method selection assistance apparatus 10 includes a processor, a memory, and a storage device as a configuration not to be illustrated. In addition, the storage device stores a computer program in which processing of a treatment-method selection assistance method according to the present example embodiment is implemented. The processor reads the computer program from the storage device into the memory and executes the computer program. Thus, the processor achieves the functions of the medication history acquisition unit 11, the medical condition acquisition unit 12, and the display control unit 13.

Alternatively, each of the medication history acquisition unit 11, the medical condition acquisition unit 12, and the display control unit 13 may be achieved by dedicated hardware. In addition, some or all of the components of each apparatus may be achieved by a general-purpose or dedicated circuitry, a processor, or the like, or a combination thereof. These may be constituted by a single chip, or may be constituted by a plurality of chips to be connected via a bus. Some or all of the components of each apparatus may be achieved by a combination of the above-described circuitry or the like and a program. Further, as the processor, a central processing unit (CPU), a graphics processing unit (GPU), a field-programmable gate array (FPGA), a quantum processor (a quantum computer control chip), and the like can be used.

In addition, in a case where some or all of the components of the treatment-method selection assistance apparatus 10 are achieved by a plurality of information processing apparatuses, circuitries, or the like, the plurality of information processing apparatuses, circuitries, or the like may be centrally arranged or distributed. For example, each of the information processing apparatus, the circuitry, and the like may be achieved as a form in which each of a client server system, a cloud computing system, and the like is connected thereto via a communication network. In addition, the functions of the treatment-method selection assistance apparatus 10 may be provided in software as a service (SaaS) format.

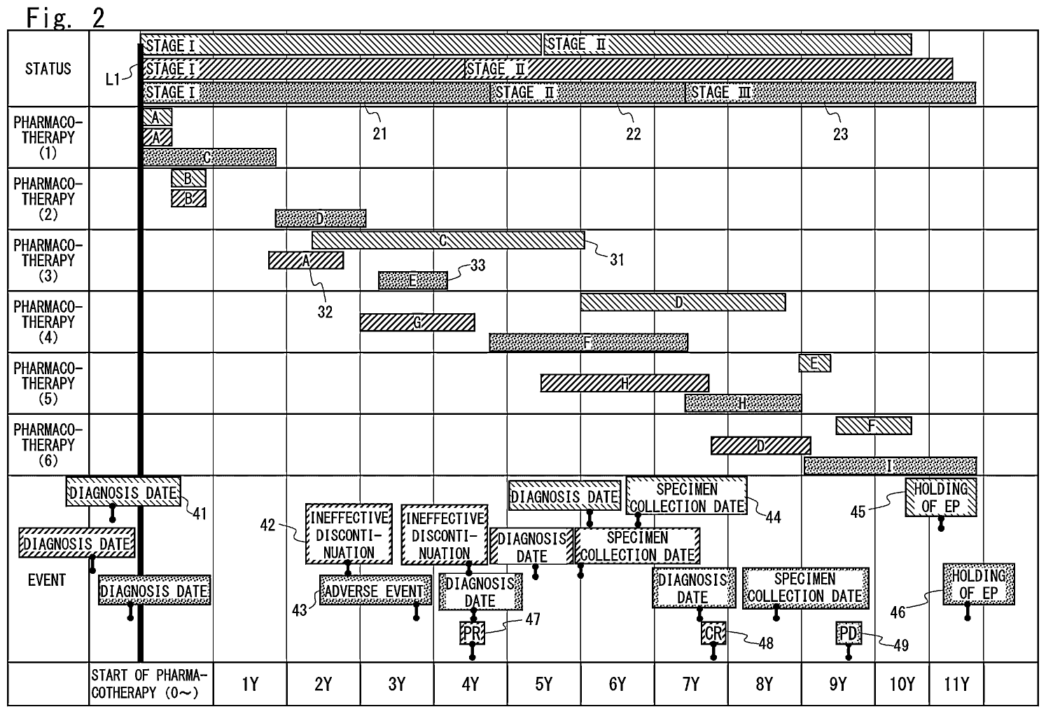

Next, with reference to FIGS. 2 to 6, a screen to be displayed by the treatment-method selection assistance apparatus 10 will be specifically explained. FIG. 2 is a schematic diagram illustrating a screen to be displayed by the display control unit 13. In FIG. 2, a lateral direction represents a time direction, and a time axis represents a time period from the start of pharmacotherapy (e.g., total days). A longitudinally extending line L1 indicates when pharmacotherapy is started. Y indicates a unit of a scale on the time axis, and a time period from the start of the pharmacotherapy is represented by 1 Y to 11 Y. The scale of the time axis, i.e., a unit of an auxiliary line, is optional and may be one week, one month, or one year. The display control unit 13 may have a function of switching the unit of the auxiliary line.

In FIG. 2, medical conditions (also referred to as status) of patients 1, 2, and 3, medication histories of the patients 1, 2, and 3, and marks indicating events are arranged vertically side by side.

First, the medical conditions of the patients 1, 2, and 3 will be explained. In FIG. 2, the medical condition of the patient 1, the medical condition of the patient 2, and the medical condition of the patient 3 are vertically arranged side by side. Each rectangular portion represents a time period during which the patient's cancer is at a constant degree of progression. For example, a rectangular portion 21 represents a time period in which the degree of progression of the cancer of the patient 3 is stage I, a rectangular portion 22 represents a time period in which the degree of progression of the cancer of the patient 3 is stage II, and a rectangular portion 23 represents a time period in which the degree of progression of the cancer of the patient 3 is stage III. Note that I represents Roman numeral 1, II represents Roman numeral 2, and III represents Roman numeral 3.

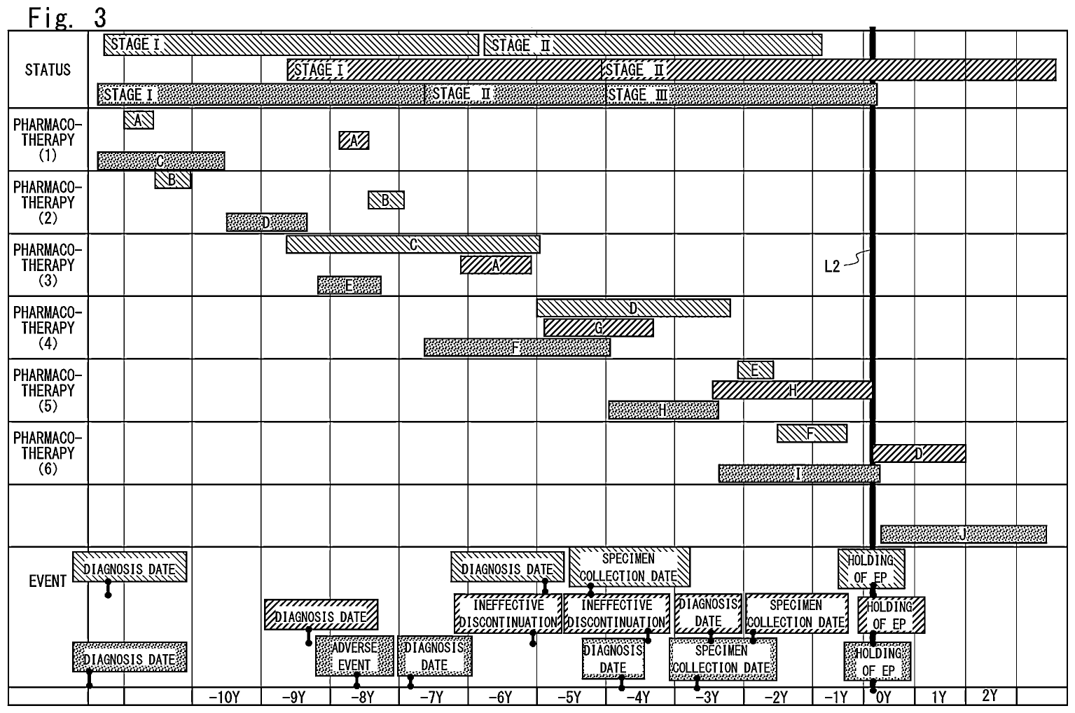

Next, the medication histories of the patients 1, 2, and 3 will be explained. Drugs A to I represent medicines to be administered. In the medication histories of FIG. 3, histories of the first to sixth pharmacotherapies are vertically arranged side by side. For example, the history of the third pharmacotherapy includes a rectangular portion 31, a rectangular portion 32, and a rectangular portion 33. The rectangular portion 31 represents a time period during which the drug C has been administered to the patient 1, the rectangular portion 32 represents a time period during which the drug A has been administered to the patient 2, and the rectangular portion 33 represents a time period during which the drug E has been administered to the patient 3.

Next, a mark indicating an event will be explained. Each mark is arranged at a position associated to an occurrence date of an event. Each mark includes a rectangular portion and a straight portion extending downward from the rectangular portion. Characters described in the rectangular portion indicate a name of the event, the above-described degree of response, and the like. The straight line portion represents a detailed position of the occurrence date of the event. Hereinafter, marks 41 to 49 among the plurality of marks will be explained in detail.

The mark 41 indicates that a diagnosis has been made on the patient 1. The mark 42 indicates that the third pharmacotherapy of the patient 2 using the drug A has been discontinued as being ineffective. The mark 43 indicates that an adverse event has occurred in the third pharmacotherapy of the patient 3 using drug E.

The mark 44 indicates that a specimen of the patient 1 has been collected. The specimen is used for cancer gene panel inspection. The mark 45 indicates holding of an expert panel for the patient 1 and the mark 46 indicates holding of an expert panel for the patient 3. The expert panel is also referred to as an EP.

The marks 47 and 48 indicate events related to the degree of response of the patient 2. In addition, the mark 47 indicates that the degree of response has been partial response (PR) and the mark 48 indicates that the degree of response has been complete response (CR). The mark 49 indicates an event related to the degree of response of the patient 3, which indicates that the degree of response has been progression (PD).

In FIG. 2, treatment stages of a plurality of patients are displayed together with the start date of the pharmacotherapy as a starting point. An appropriate treatment policy can be selected by confirming a treatment effect and a response rate between patients (e.g., cancer patients).

In the screen illustrated in FIG. 2, the time axis indicates a time period elapsed from the start of the pharmacotherapy, but as described above, the time axis may indicate the number of elapsed days from another event. In FIG. 3, the time axis represents an elapsed time period from the date of holding of the expert panel. Except for this, it is the same as in FIG. 2, and explanation of the same parts as in FIG. 2 will be omitted. A line L2 extending in a longitudinal direction indicates the date of holding of the expert panel. −10 Y to 2 Y represent an elapsed time period from the date of holding of the expert panel. Minus of the sign represents that the event has been performed before the expert panel.

In FIG. 3, the start date of the expert panel is used as a starting point. Therefore, it is possible to visualize how the treatment policy has changed before and after the start of the expert panel, i.e., after cancer genome information is taken into account. In addition, information regarding whether a medical condition has improved or deteriorated after a certain medicine is prescribed is also visualized.

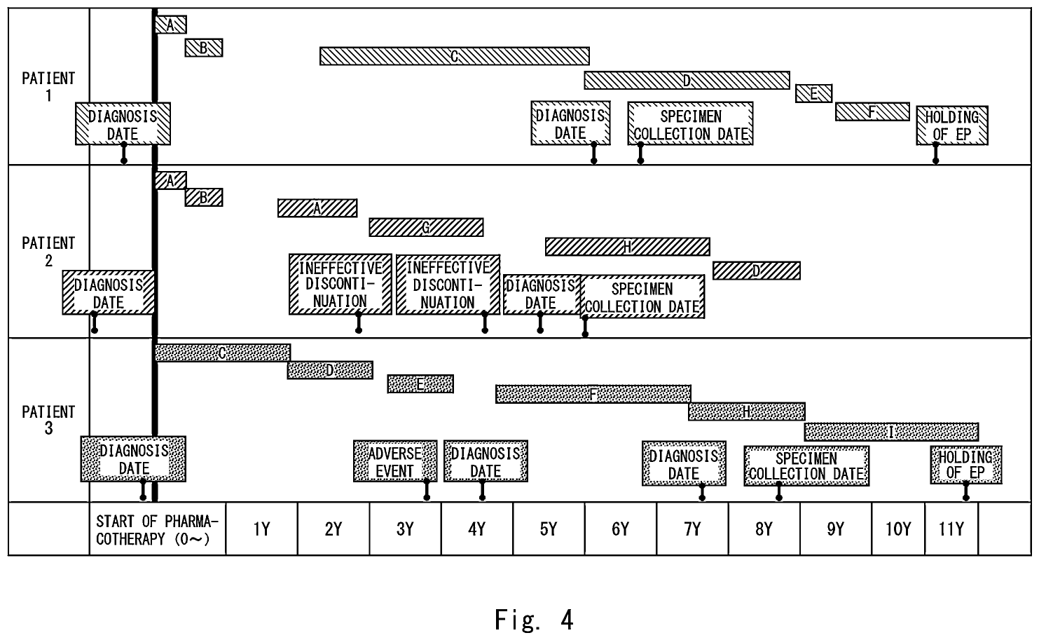

On the screens illustrated in FIG. 2 and FIG. 3, the medication history, the medical condition, and the event of each patient are separately displayed. However, the display control unit 13 may display information collectively for each patient. FIG. 4 illustrates a case in which information of each patient is collectively displayed. Except for this, it is the same as in FIG. 2, and explanation of the same parts as in FIG. 2 will be omitted.

In FIG. 4, the information regarding the patient 1, the information regarding the patient 2, and the information regarding the patient 3 are vertically arranged side by side. In addition, the medication history and the event for each patient are vertically displayed side by side. In FIG. 4, the display of the medical condition is omitted.

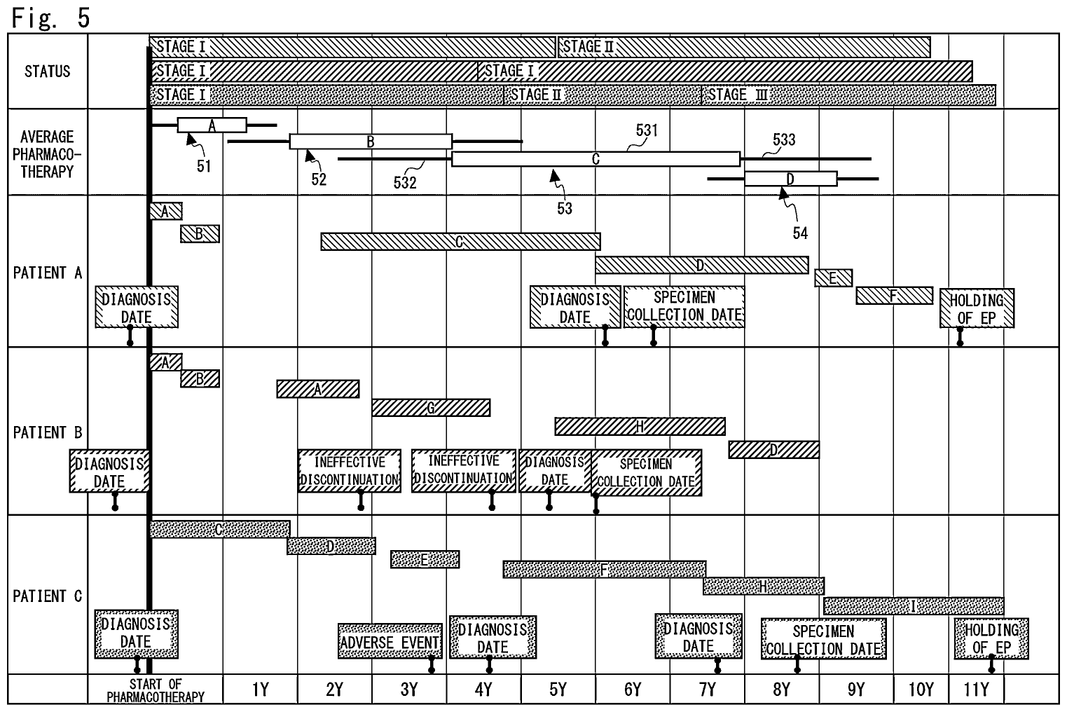

The screen to be displayed may include a chart illustrating an average pharmacotherapy flow. In FIG. 5, graphics 51, 52, 53, and 54 are added as compared with FIG. 4. Except for this, it is the same as in FIG. 2, and explanation of the same part as in FIG. 2 will be omitted.

The graphics 51, 52, 53, and 54 indicate statistical information relating to medication periods of drugs A, B, C, and D, respectively.

Each of the graphics 51 to 54 includes a rectangular portion and a straight portion. For example, the graphic 53 includes a rectangular portion 531 and straight portions 532 and 533. The rectangular portion 531 indicates an average medication period of the drug C. A time period from a left end of the straight portion 532 to a right end of the straight portion 533 represents the maximum value of the medication period of the drug C.

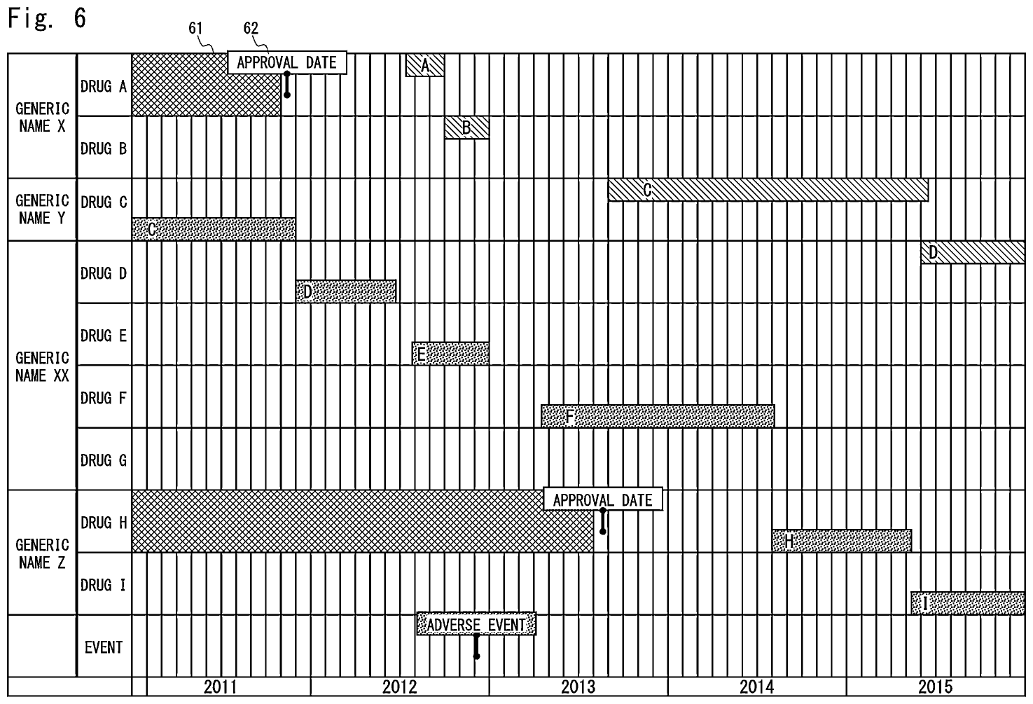

Next, a case of displaying an unapproved time period of a medicine will be explained with reference to FIG. 6. In the screen illustrated in FIG. 6, the time axis represents a date. 2011 to 2015 represent the year of the Christian era, and the unit of the auxiliary line represents one month. In FIG. 6, the display of the medical condition is omitted similarly to FIG. 4 and the like. Hereinafter, explanation of the same parts as in FIG. 2 will be omitted.

In the medication history of FIG. 6, patients to whom the same medicine has been administered are collectively displayed. A medication history of a medicine represented by a generic name X, a medication history of a medicine represented by a generic name Y, a medication history of a medicine represented by a generic name XX, and a medication history of a medicine represented by a generic name Z are arranged vertically side by side. The medicine represented by the generic name X includes the drug A and the drug B. The medicine represented by the generic name Y includes the drug C. The medicine represented by the generic name XX includes the drugs D to G. The medicine represented by the generic name Z includes the drug H and the drug I. For example, the medication history of the drug C includes the medication history of the patient 1 and the medication history of the patient 3.

The rectangular portion 61 indicates a time period during which the drug A is not approved. A mark 62 indicates an approval date of the drug A. Similarly, a time period during which the drug H is not approved and an approval date of the drug H are indicated. In FIG. 6, an adverse event is displayed as an event, but a reason for invalidation may be further displayed.

Finally, effects achieved by the first example embodiment will be explained. The treatment-method selection assistance apparatus according to the first example embodiment displays medication histories and medical conditions of a plurality of patients on a time axis. The treatment-method selection assistance apparatus 10 can assist a physician to select a treatment method by displaying information regarding a plurality of patients.

In the examples described above, the program includes instructions (or software code) that, when loaded into a computer, cause a computer to perform one or more of the functions explained in the example embodiments. The program may be stored in a non-transitory computer-readable medium or a tangible storage medium. By way of example, and not limitation, computer-readable media or tangible storage media include a random-access memory (RAM), a read-only memory (ROM), a flash memory, a solid-state drive (SSD) or other memory techniques, a CD-ROM, a digital versatile disc (DVD), a Blu-ray (registered trademark) disc or other optical disc storage, magnetic cassettes, magnetic tape, a magnetic disk storage or other magnetic storage devices. The program may be transmitted on a transitory computer-readable medium or a communication medium. By way of example, and not limitation, transitory computer-readable media or communication media include electrical, optical, acoustic, or other forms of propagated signals.

Note that the present disclosure is not limited to the above-described example embodiments, and can be appropriately modified without departing from the spirit thereof. Further, the present disclosure may be implemented by appropriately combining the example embodiments.

Although the present invention has been explained with reference to the example embodiments, the present invention is not limited to the above example embodiments. Various modifications that can be understood by a person skilled in the art within the scope of the present invention can be made to the configuration and details of the present invention.

### REFERENCE SIGNS LIST

10 TREATMENT-METHOD SELECTION ASSISTANCE APPARATUS
11 MEDICATION HISTORY ACQUISITION UNIT
12 MEDICAL CONDITION ACQUISITION UNIT
13 DISPLAY CONTROL UNIT
1, 2, 3 PATIENT
A, B, C, D, E, F, G, H, I DRUG
21, 22, 23, 31, 32, 33, 531, 61 RECTANGULAR PORTION
41, 42, 43, 44, 45, 46, 47, 48, 49, 62 MARK
51, 52, 53, 54 GRAPHIC
532, 533 STRAIGHT PORTION
L1, L2 STRAIGHT LINE

1. A treatment-method selection assistance apparatus comprising:
at least one memory storing instructions and
at least one processor configured to execute the instructions to:
acquire a history of administering a medicine to each patient as a medication history;
acquire a state of a disease of each patient; and
cause a display apparatus to display a screen on which the medication histories and states of the disease of a plurality of patients are displayed along a time axis.

2. The treatment-method selection assistance apparatus according to claim 1, wherein the at least one processor is further configured to execute the instructions to:
cause the screen to display an event in which a date is determined for each patient.

3. The treatment-method selection assistance apparatus according to claim 2, wherein the at least one processor is further configured to execute the instructions to:
switch between a first screen in which the time axis represents a date and a second screen in which the time axis represents a time period from the event, according to a user's selection.

4. The treatment-method selection assistance apparatus according to claim 2, wherein the event is at least one of starting or ending of administration of the medicine and holding of an expert panel for each patient.

5. The treatment-method selection assistance apparatus according to claim 2, wherein
the event is related to a degree of response indicating a treatment effect for each patient, and
the at least one processor is further configured to execute the instructions to:
display the degree of response on the screen.

6. The treatment-method selection assistance apparatus according to claim 1, wherein the at least one processor is further configured to execute the instructions to:
display, on the screen, a time period during which the medicine is not approved.

7. The treatment-method selection assistance apparatus according to claim 1, wherein the state of the disease is any one of cancer progression, malignancy, class classification, and prognostic factors.

8. The treatment-method selection assistance apparatus according to claim 1, wherein the at least one processor is further configured to execute the instructions to:
display, on the screen, at least one of a medical history of each patient, a medical history of a family of each patient, and gene mutation information regarding each patient.

9. A treatment-method selection assistance method comprising by a computer:
acquiring a history of administering a medicine to each patient as a medication history;
acquiring a state of a disease of each patient; and
causing a display apparatus to display a screen on which the histories and states of the disease of a plurality of patients are displayed along a time axis.

10. A non-transitory computer-readable medium storing a program for causing a computer to execute:
processing of acquiring a history of administering a medicine to each patient as a medication history;
processing of acquiring a state of a disease of each patient; and
processing of causing a display apparatus to display a display screen on which the histories and states of the disease of a plurality of patients are displayed along a time axis.
